(19) United States
(12) Patent Application Publication
(10) Pub. No.: US 2007/0037777 A1
(43) Pub. Date: Feb. 15, 2007

(54) Lipid-containing compositions and methods of using them

(75) Inventor: Leonard S. Girsh, Naples, FL (US)

Correspondence Address: IP GROUP OF DLA PIPER US LLP, ONE LIBERTY PLACE, 1650 MARKET ST, SUITE 4900, PHILADELPHIA, PA, 19103, US

(73) Assignee: Immunopath Profile, Inc.

(21) Appl. No.: 11/501,380

(22) Filed: Aug. 9, 2006

Related U.S. Application Data
(60) Provisional application No. 60/707,571, filed on Aug. 12, 2005.

(51) Int. Cl. A61K 31/198; A61K 31/685; A61K 47/00

(52) U.S. Cl. 514/78; 514/561; 514/773; 514/785

(57) ABSTRACT

Anabolic compositions can be administered to patients with chronic diseases, or who suffer from conditions precipitated by such diseases or long term treatment regimes. The compositions comprise at least one surfactant wherein the total concentration of surfactant in the composition is greater than 1% (w/w or w/v), and can cause weight gain in chronic disease patients as well as reduce or eliminate other disease symptoms or conditions. The anabolic compositions can also provide therapeutic benefit in patients who are candidates for, or who have undergone, organ or tissue transplant procedures.

### RELATED APPLICATIONS

This application claims the benefit of co-pending U.S. provisional application 60/707,571, filed on Aug. 12, 2005, the entire disclosure of which is herein incorporated by reference.

### FIELD OF THE INVENTION

This invention relates to the field of nutritional therapy for diseases, for example chronic diseases such as cancer. The therapy comprises administration of a composition comprising a certain amount of lipid, optionally together with other components such as amino acids, extracellular matrix components, anti-inflammatories, and the like.

### BACKGROUND

Anorexia/cachexia syndrome, along with dehydration and oxidative stress, plays a key role in the progression and outcome of neoplastic and other diseases. The severity of cachexia in many illnesses can be the primary determining factor in both quality of life and eventual mortality.

Cachexia is commonly observed in patients with cancer, particularly in children and elderly individuals. Cachexia can also be present in other clinical situations, such as in patients recovering from surgery, trauma, AIDS related complications, patients awaiting chemotherapy and failure to thrive end stage disease. For example, at the time of diagnosis, 80% of patients with upper gastrointestinal cancers and 60% of patients with lung cancer have already experienced substantial weight loss. The characteristic clinical picture of anorexia, tissue wasting, loss of body weight accompanied by a decrease in muscle mass and adipose tissue, and poor “performance status” that often precedes death has been named cancer-related anorexia/cachexia syndrome.

The prevalence of cachexia in neoplastic disease increases from 50% to 80% before death, and cachexia is the main cause of death in 20% of cancer patients.

Cachexia and dehydration in cancer patients is linked to abnormalities in carbohydrate, protein, and lipid biochemistry/metabolism, and changes in energy metabolism. Circulating factors produced by a tumor or by the host immune system in response to the tumor, such as cytokines released by lymphocytes or monocyte/macrophages, may also contribute to cachexia and dehydration. For example, several pro-inflammatory cytokines, including interleukin (IL)-1, IL-6, tumor necrosis factor-alpha, IFN-alpha, and IFN-gamma, have been implicated in the pathogenesis of cachexia associated with human cancers.

Additional factors and mechanisms thought to play a central role in cachexia and dehydration are the presence of a chronic systemic inflammatory state, circulating tumor-derived lipolytic and proteolytic factors, increased futile energy consuming cycles, and a decreased food intake. The resulting malnutrition and loss of lean body mass with concurrent unrecognized dehydration status reduces the quality of life, decreases tolerance to therapy and increases post surgical and/or oncological complications.

Attempts to treat cachexia with a variety of agents have met with limited success. The most widely used agent, megestrol acetate, has shown some promise in reversing weight loss. However, this is primarily attributable to increases in fat mass and water retention, rather than preservation of lean body mass. Steroids have also been used to increase blood glucose levels in an attempt to reverse weight loss. Such drug therapies used to treat cachexia are not well tolerated by many patients.

Traditional nutritional supplementation therapies have limited effect, or even serve to aggravate the cachexia in cancer patients. For example, patients who are force-fed high glucose formulas which contain 50% or more refined corn sweeteners (such as Boost or Ensure) show increased metabolic activity of malignant cells observed by PET scan.

Other disease states, in particular chronic diseases, may also benefit from nutritional supplementation which encourages an anabolic environment while not aggravating (or even mitigating) the patient's condition. For example, chronic gastro-intestinal conditions such as inflammatory bowel disease or Crohn's disease can be treated with high-energy nutritional supplements. However, current nutritional therapies are typically administered with anti-inflammatory drugs such as corticosteroids, which interfere with the patient's health and ability to heal tissue in the long term. Metabolic storage diseases can also be treated with nutritional supplementation or altered diet, but such diets can be difficult to maintain, and are often not entirely effective. Tissue damage often occurs in patients with chronic disease, or is a sequelae to long-term treatment of such diseases.

What is needed, therefore, is a composition and method of using it that can treat cachexia and other conditions associated with chronic disease, where the composition is both effective and well-tolerated by patients. Desirably, the composition and methods of its use does not exacerbate the disease or disease symptoms, and can even improve the patient's condition.

### SUMMARY

Anabolic compositions can be administered to patients with chronic diseases, or who suffer from conditions precipitated by such diseases or long term treatment regimes. It has been discovered that such compositions comprising a certain amount of surfactant (such as a lipid) can cause weight gain in chronic disease patients, and can also reduce or eliminate other disease symptoms or conditions being experienced by these patients. The anabolic compositions can also provide therapeutic benefit in patients who are candidates for, or who have undergone, organ or tissue transplant procedures.

In one aspect, there is provided an anabolic composition, comprising at least one amino acid, at least one extracellular matrix compound, and at least one surfactant, wherein the concentration of surfactant in the composition is about 1% or greater (w/w or w/v) with respect to the total composition.

In another aspect, there is provided a method of treating a disease or condition in a subject, comprising providing a subject who has the disease or condition, and administering to the subject an effective amount of a composition comprising at least one surfactant, wherein the total concentration of surfactant in the composition is greater than 1% (w/w or w/v).

### DETAILED DISCLOSURE

An anabolic composition is provided, which comprises at least one amino acid, at least one extracellular matrix compound, and at least one surfactant, wherein the concentration of surfactant in the composition is about 1% or greater with respect to the total composition. All percentages disclosed herein refer either to weight to volume (if a liquid composition) or weight to weight (if a solid composition).

Any biocompatible surfactant can be used in the composition. Such surfactants are known to those of skill in the art, and representative examples are included in Table 1 below. In addition to the surfactants listed in Table 1, suitable surfactants for use in the composition include lipids (for example phospholipids such as phosphatidylcholine, phosphatidylserine, phosphatidylinositol, phosphatidylethanolamine, phosphatidic acid and phosphotidyl glycerol); essential lipids (which can contain linoleic and linolenic acids), DHA, EPA, sphingolipids; sphingomyelin; glycolipids; cerbrosides; gangliosides; cephalin; lipovitellin; glycosphingolipids; and combinations thereof, monoglycerides, diglycerides, lipoproteins; polyglycerol polyricinolate; polysorbate 80; polysorbate 65 and sodium lauryl sulfate; and combinations thereof.

The total amount of surfactant present in the composition can be in an amount of about 1% or greater; for example between about 1.2% and about 20%. Suitable amounts of surfactant in the composition include about 1.2%, about 1.3%, about 1.4%, about 1.5%, about 1.6%, about 1.7%, about 1.8%, about 1.9%, about 2%, about 3%, about 4%, about 5%, about 6%, about 7%, about 7.5%, about 8%, about 9%, about 10%, about 11%, about 12%, about 13%, about 14%, about 15%, about 16%, about 17%, about 18%, about 19%, about 20%. Amounts of surfactant greater than about 20% (e.g., about 25%, about 30%, about 35%, about 40%, about 45% or about 50%) are contemplated.

In some embodiments, the composition can comprise phosphotidylcholine in amounts of about 19% (to mimic human red blood cell membrane), about 10% (to mimic myelin membrane), about 39% (to mimic heart mitochondrial membrane). In some embodiments, the composition can comprise phosphotidylethanolamine in amounts of about 18% (to mimic human red blood cell membrane), about 20% (to mimic myelin membrane) and about 27% (to mimic heart mitochondrial membrane). In some embodiments, the composition can comprise phosphotidylinositol in amounts of about 1% (to mimic human red blood cell membrane), about 1% (to mimic myelin membrane) and about 7% (to mimic heart mitochondrial membrane). In some embodiments, the composition can comprise phosphotidylserine in amounts of about 8% (to mimic human red blood cell membrane), about 8% (to mimic myelin membrane) and about 0.5% (to mimic heart mitochondrial membrane). In some embodiments, the composition can comprise phosphotidylserine in amounts of about 18% (to mimic human red blood cell membrane), about 20% (to mimic myelin membrane) and about 27% (to mimic heart mitochondrial membrane). In some embodiments, the composition can comprise sphingomyelin in amounts of about 17.5% (to mimic human red blood cell membrane), about 8.5% (to mimic myelin membrane) and about 0% (to mimic heart mitochondrial membrane). In some embodiments, the composition can comprise glycolipid in amounts of about 10% (to mimic human red blood cell membrane), about 26% (to mimic myelin membrane) and about 0% (to mimic heart mitochondrial membrane). In some embodiments, the composition can comprise phosphatidic acid in amounts of about 1.5% (to mimic human red blood cell membrane), about 0.5% (to mimic myelin membrane), about 0% (to mimic heart mitochondrial membrane). In some embodiments, the composition can comprise phosphotidylglycerol in amounts of about 0% (to mimic human red blood cell membrane), about 0% (to mimic myelin membrane), about 0% (to mimic heart mitochondrial membrane). In contrast, the E. coli cell membrane has 0% phosphotidylcholine, 0% phosphotidylinositol, 0% phosphotidylserine, 0% sphingomyelin, 0% glycolipid, 0% phosphatidic acid, 18% phosphotidylglycerol and 65% phosphotidylethanolamine.

The at least one surfactant in the composition can have hydrophilic/lipophilic balance (“HLB”) of less than about six (e.g., about 1, about 2, about 3, about 4, about 5), or an HLB of about six or greater (e.g., about 7, about 8, about 9, about 10, about 11, about 12, about 13, about 14, about 15, about 16, about 17, about 18, about 19 or about 20). In one embodiment, the surfactant has an HLB value of about 13.

TABLE 1
Exemplary Surfactants

| Name | Mfr.* | Chemical Designation | Type† | HLB†† |
|---|---|---|---|---|
| Span 85 | 1 | Sorbitan trioleate | N | 1.8 |
| Arlacel 85 | 1 | Sorbitan trioleate | N | 1.8 |
| Atlas G-1706 | 1 | Polyoxyethylene sorbitol beeswax | N | 2 |
| | | derivative | | |
| Span 85 | 1 | Sorbitan tristearate | N | 2.1 |
| Arlacel 65 | 1 | Sorbitan tristearate | N | 2.1 |
| Atlas G-1050 | 1 | Polyoxyethylene sorbitol hexastearate | N | 2.6 |
| Emcol EO-50 | 2 | Ethylene glycol fatty acid ester | N | 2.7 |
| Emcol ES-50 | 2 | Ethylene glycol fatty acid ester | N | 2.7 |
| Atlas G-1704 | 1 | Polyoxyethylene sorbitol beeswax | N | 3 |
| | | derivative | | |
| Emeol PO-50 | 2 | Propylene glycol fatty acid ester | N | 3.4 |
| Atlas G-922 | 1 | Propylene glycol monostearate | N | 3.4 |
| “Pure” | 6 | Propylene glycol monostearate | N | 3.4 |
| Atlas G-2158 | 1 | Propylene glycol monostearate | N | 3.4 |
| Emcol PS-50 | 2 | Propylene glycol fatty acid ester | N | 3.4 |
| Emcol EL-50 | 2 | Ethylene glycol fatty acid ester | N | 3.6 |
| Emcol PP-50 | 2 | Propylene glycol fatty acid ester | N | 3.7 |
| Arlacel C | 1 | Sorbitan sesquioleate | N | 3.7 |
| Arlacel 83 | 1 | Sorbitan sesquioleate | N | 3.7 |
| Atlas G-2859 | 1 | Polyoxyethylene sorbitol 4.5 oleate | N | 3.7 |
| Atmul 67 | 1 | Glycerol monostearate | N | 3.8 |
| Atmul 84 | 1 | Glycerol monostearate | N | 3.8 |
| Tegin 515 | 5 | Glycerol monostearate | N | 3.8 |
| Aldo 33 | 4 | Glycerol monostearate | N | 3.8 |
| “Pure” | 6 | Glycerol monostearate | N | 3.8 |
| Atlas G-1727 | 1 | Polyoxyethylene sorbitol beeswax | N | 4 |
| | | derivative | | |
| Emcol PM-50 | 2 | Propylene glycol fatty acid ester | N | 4.1 |
| Span 80 | 1 | Sorbitan monooleate | N | 4.3 |
| Arlacel 80 | 1 | Sorbitan monooleate | N | 4.3 |
| Atlas G-917 | 1 | Propylene glycol monolaurate | N | 4.5 |
| Atlas G-3851 | 1 | Propylene glycol monolaurate | N | 4.5 |
| Emcol PL-50 | 2 | Propylene glycol fatty acid ester | N | 4.5 |
| Span 60 | 1 | Sorbitan monostearate | N | 4.7 |
| Arlacel 60 | 1 | Sorbitan monostearate | N | 4.7 |
| Atlas G-2139 | 1 | Diethylene glycol monooleate | N | 4.7 |
| Emcol DO-50 | 2 | Diethylene glycol fatty acid ester | N | 4.7 |
| Atlas G-2146 | 1 | Diethylene glycol monostearate | N | 4.7 |
| Emcol DS-50 | 2 | Diethylene glycol fatty acid ester | N | 4.7 |
| Atlas G-1702 | 1 | Polyoxyethylene sorbitol beeswax | N | 5 |
| | | derivative | | |
| Emcol DP-50 | 2 | Diethylene glycol fatty acid ester | N | 5.1 |
| Aldo 28 | 4 | Glycerol monostearate (self- | A | 5.5 |
| | | emulsifying) | | |
| Tegin | 5 | Glycerol monostearate (self- | A | 5.5 |
| | | emulsifying) | | |
| Emcol DM-50 | 2 | Diethylene glycol fatty acid ester | N | 5.6 |
| Atlas G-1725 | 1 | Polyoxyethylene sorbitol beeswax | N | 6 |
| | | derivative | | |
| Atlas G-2124 | 1 | Diethylene glycol monolaurate (soap | N | 6.1 |
| | | free) | | |
| Emcol DL-50 | 2 | Diethylene glycol fatty acid ester | N | 6.1 |
| Glaurin | 4 | Diethylene glycol monolaurate (soap | N | 6.5 |
| | | free) | | |
| Span 40 | 1 | Sorbitan monopalmitate | N | 6.7 |
| Arlacel 40 | 1 | Sorbitan monopalmitate | N | 6.7 |
| Atlas G-2242 | 1 | Polyoxyethylene dioleate | N | 7.5 |
| Atlas G-2147 | 1 | Tetraethylene glycol monostearate | N | 7.7 |
| Atlas G-2140 | 1 | Tetraethylene glycol monooleate | N | 7.7 |
| Atlas G-2800 | 1 | Polyoxypropylene mannitol dioleate | N | 8 |
| Atlas G-1493 | 1 | Polyoxyethylene sorbitol lanolin oleate | N | 8 |
| | | derivative | | |
| Atlas G-1425 | 1 | Polyoxyethylene sorbitol lanolin | N | 8 |
| | | derivative | | |
| Atlas G-3608 | 1 | Polyoxypropylene stearate | N | 8 |
| Span 20 | 1 | Sorbitan monolaurate | N | 8.6 |
| Arlacel 20 | 1 | Sorbitan monolaurate | N | 8.6 |
| Emulphor VN-430 | 3 | Polyoxyethylene fatty acid | N | 9 |
| Atlas G-1734 | 1 | Polyoxyethylene sorbitol beeswax | N | 9 |
| | | derivative | | |
| Atlas G-2111 | 1 | Polyoxyethylene oxypropylene oleate | N | 9 |
| Atlas G-2125 | 1 | Tetraethylene glycol monolaurate | N | 9.4 |
| Brij 30 | 1 | Polyoxyethylene lauryl ether | N | 9.5 |
| Tween 61 | 1 | Polyoxyethylene sorbitan monostearate | N | 9.6 |
| Atlas G-2154 | 1 | Hexaethylene glycol monostearate | N | 9.6 |
| Tween 81 | 1 | Polyoxyethylene sorbitan monooleate | N | 10.0 |
| Atlas G-1218 | 1 | Polyoxyethylene esters of mixed fatty | N | 10.2 |
| | | and resin acids | | |
| Atlas G-3806 | 1 | Polyoxyethylene cetyl ether | N | 10.3 |
| Tween 65 | 1 | Polyoxyethylene sorbitan tristearate | N | 10.5 |
| Atlas G-3705 | 1 | Polyoxyethylene lauryl ether | N | 10.8 |
| Tween 85 | 1 | Polyoxyethylene sorbitan trioleate | N | 11 |
| Atlas G-2116 | 1 | Polyoxyethylene oxypropylene oleate | N | 11 |
| Atlas G-1790 | 1 | Polyoxyethylene lanolin derivative | N | 11 |
| Atlas G-2142 | 1 | Polyoxyethylene monooleate | N | 11.1 |
| Myrj 45 | 1 | Polyoxyethylene monostearate | N | 11.1 |
| Atlas G-2141 | 1 | Polyoxyethylene monooleate | N | 11.4 |
| P.E.G. 400 monooleate | 6 | Polyoxyethylene monooleate | N | 11.4 |
| P.E.G. 400 monooleate | 7 | Polyoxyethylene monooleate | N | 11.4 |
| Atlas G-2076 | 1 | Polyoxyethylene monopalmitate | N | 11.6 |
| S-541 | 4 | Polyoxyethylene monostearate | N | 11.6 |
| P.E.G. 400 monostearate | 6 | Polyoxyethylene monostearate | N | 11.6 |
| P.E.G. 400 monostearate | 7 | Polyoxyethylene monostearate | N | 11.6 |
| Atlas G-3300 | 1 | Alkyl aryl sultanate | A | 11.7 |
| | | Triethanolamine oleate | A | 12 |
| Atlas G-2127 | 1 | Polyoxyethylene monolaurate | N | 12.8 |
| Igepal CA-630 | 3 | Polyoxyethylene alkyl phenol | N | 12.8 |
| Atlas G-1431 | 1 | Polyoxyethylene sorbitol lanolin | N | 13 |
| | | derivative | | |
| Atlas G-1690 | 1 | Polyoxyethylene alkyl aryl ether | N | 13 |
| S-307 | 4 | Polyoxyethylene monolaurate | N | 13.1 |
| P.E.G. 400 monolaurate | 6 | Polyoxyethylene monolaurate | N | 13.1 |
| Atlas G-2133 | 1 | Polyoxyethylene, lauryl ether | N | 13.1 |
| Atlas G-1794 | 1 | Polyoxyethylene castor oil | N | 13.3 |
| Emulphor EL-719 | 3 | Polyoxyethylene vegetable oil | N | 13.3 |
| Tween 21 | 1 | Polyoxyethylene sorbitan monolaurate | N | 13.3 |
| Renex 20 | 1 | Polyoxyethylene esters of mixed fatty | N | 13.5 |
| | | and resin acids | | |
| Atlas G-1441 | 1 | Polyoxyethylene sorbitol lanolin | N | 14 |
| | | derivative | | |
| Atlas G-7596J | 1 | Polyoxyethylene sorbitan monolaurate | N | 14.9 |
| Tween 60 | 1 | Polyoxyethylene sorbitan monostearate | N | 14.9 |
| Tween 80 | 1 | Polyoxyethylene sorbitan monooleate | N | 15 |
| Myrj 49 | 1 | Polyoxyethylene monostearate | N | 15.0 |
| Atlas G-2144 | 1 | Polyoxyethylene monooleate | N | 15.1 |
| Atlas G-3915 | 1 | Polyoxyethylene oleyl ether | N | 15.3 |
| Atlas G-3720 | 1 | Polyoxyethylene stearyl alcohol | N | 15.3 |
| Atlas G-3920 | 1 | Polyoxyethylene oleyl alcohol | N | 15.4 |
| Emulphor ON-870 | 3 | Polyoxyethylene fatty alcohol | N | 15.4 |
| Atlas G-2079 | 1 | Polyoxyethylene glycol monopalmitate | N | 15.5 |
| Tween 40 | 1 | Polyoxyethylene sorbitan | N | 15.6 |
| | | monopalmitate | | |
| Atlas G-3820 | 1 | Polyoxyethylene cetyl alcohol | N | 15.7 |
| Atlas G-2162 | 1 | Polyoxyethylene oxypropylene stearate | N | 15.7 |
| Atlas G-1471 | 1 | Polyoxyethylene sorbitol lanolin | N | 16 |
| | | derivative | | |
| Myrj 51 | 1 | Polyoxyethylene mono stearate | N | 16.0 |
| Atlas G-7596P | 1 | Polyoxyethylene sorbitan monolaurate | N | 16.3 |
| Atlas G-2129 | 1 | Polyoxyethylene monolaurate | N | 16.3 |
| Atlas G-3930 | 1 | Polyoxyethylene oleyl ether | N | 16.6 |
| Tween 20 | 1 | Polyoxyethylene sorbitan monolaurate | N | 16.7 |
| Brij 35 | 1 | Polyoxyethylene lauryl ether | N | 16.9 |
| Myrj 52 | 1 | Polyoxyethylene monostearate | N | 16.9 |
| Myrj 53 | 1 | Polyoxyethylene monostearate | N | 17.9 |
| | | Sodium oleate | A | 18 |
| Atlas G-2159 | 1 | Polyoxyethylene monostearate | N | 18.8 |
| | | Potassium oleate | A | 20 |
| Atlas G-263 | 1 | N-cetyl N-ethyl morpholinium | C | 25-30 |
| | | ethosulfate | | |
| | | Pure sodium lauryl sulfate | A | App. 40 |

*1 = Atlas Powder Company, 2 = Emulsol Corporation, 3 = General Aniline & Film Corporation, 4 = Glyco Products Company, Inc., 5 = Goldschmidt Chemical Corporation, 6 = Kessler Chemical Company, Inc., 7 = W.C. Hardesty Company, Inc.
†A = Anionic, C = Cationic, N = Nonionic.
††HLB values, either calculated or determined, believed to be correct to ±1.

The composition can comprise one or more other components, such as amino acids; extracellular matrix components; electrolytes, minerals, vitamins or trace elements; and probiotics. In some embodiments, the composition can further comprise vitelloprotein.

Any amino acid or combination of amino acids can be used in the composition. For example, the 20 naturally-occurring L amino acids (and glycine, which has no stereospecificity) can be used, as the L-stereoisomer is what the mammalian body naturally makes and uses. The L amino acids can be optically pure form. “Optically pure” as used herein means having at least about 90% by weight of one stereoisomer and about 10% by weight or less of one or more other stereoisomers. For example, the L amino acids can be at least about 95% by weight of the L isomer and about 5% by weight or less of the D isomer, such as greater than about 99% by weight of the L isomer and about 1% or less by weight of the D isomer. Optically pure L amino acids are commercially available and are preferred, and also are readily obtainable by methods known to those of skill in the art, for example, by synthesis from an optically pure intermediate.

The amino acids used in the composition can comprise one or more essential amino acids. As used herein, “essential amino acids” are those amino acids that must be supplied in the diet because an organism cannot synthesize sufficient quantities of them. Essential amino acids for adult humans are arginine, histidine, isoleucine, leucine, lysine, methionine, threonine, tryptophan, and valine. Essential amino acids for other groups of human patients or other organisms are known to those of skill in the art.

The amino acids used in the composition can comprise one or more free amino acids, or can be supplied as part of a peptide or protein. As used herein, “free amino acids” are those amino acids that are not part of a peptide or a protein. Free amino acids can be in acid or salt form.

Amino acids for use in the composition can be derived from natural sources or can be synthetically produced. Suppliers of suitable amino acids include Ajinomoto USA of Torrance, Calif. and Tanabe USA Inc. of San Diego, Calif. One exemplary source of amino acids is Neocate® elemental diet, sold by SHS of Liverpool, UK, which contains inter alia essential and non-essential amino acids, dried glucose syrup, fat, minerals, trace elements and vitamins.

The amount of amino acid(s) comprising the compositions can be those daily amounts recommended as an elemental diet for infants or others suffering from gastrointestinal problems. For example, the total amino acid amount in the compositions can be less than about 20 grams, such as about 15 grams or about 10 grams. A suitable amount of amino acid(s) in the composition can comprise 1-2 grams amino acids administered as part of the composition three to four times daily, for a total amount of three to eight grams daily.

Greater or lesser amounts of amino acids in the composition are contemplated, for example about 0.5 to about 0.9 grams daily.

Other suitable amino amounts comprising the composition can be within the following weight ranges, for daily administration:

L alanine, about 0.5 to about 12.5 grams, for example about 5 to about 9 grams.

L arginine: about 0.05 to about 12.5 grams, for example about 1 to about 9 grams.

L asparagine about 0.05 to about 12.5 grams, for example about 0.5 to about 9 grams.

L aspartic acid: about 0.05 to about 6 grams, for example about 0.5 to about 6 grams.

L cysteine: about 0.1 to about 1 gram, for example about 0.5 to about 1 gram.

L cystine: about 0.5 to about 12.5 grams, for example about 0.5 to about 9 grams.

L glutamine: about 0.5 to about 12.5 grams, for example about 0.5 to about 9 grams.

L glutamic acid: about 0.5 to about 6 grams.

Glycine about 0.5 to about 12.5 grams, for example about 0.5 to about 9 grams.

L histidine about 0.5 to about 12.5 grams, for example about 0.5 to about 9 grams.

L isoleucine: about 0.5 to about 12.5 grams, for example about 1 to about 9 grams.

L leucine about 0.5 to about 12.5 grams, for example about 0.5 to about 5 grams.

L lysine: about 0.5 to about 12.5 grams, for example about 0.5 to about 9 grams.

L methionine: about 0.5 to about 12.5 grams, for example about 0.5 to about 9 grams.

L phenylalanine: about 0.5 to about 12.5 grams, for example about 0.5 to about 9 grams.

L proline: about 0.5 to about 12.5 grams, for example about 1 to about 9 grams

L serine: about 0.5 to about 6 grams.

L threonine: about 0.5 to about 12.5 grams, for example about 0.5 to about 9 grams.

L tryptophan: about 0.5 to about 6 grams.

L tyrosine: about 0.5 to about 12.5 grams, for example about 0.5 to about 9 grams.

L valine: about 0.5 to about 5 grams, for example about 0.5 to about 3 grams.

L taurine: about 0.5 to about 12.5 grams, for example about 0.5 to about 9 grams.

L carnitine: about 0.5 to about 12.5 grams, for example about 0.5 to about 9 grams.

The composition can also comprise one or more essential lipids in addition to the surfactants discussed above. As used herein, “essential lipids” are those lipids that must be supplied in the diet because an organism cannot synthesize them in sufficient quantities. For mammals, the essential lipids include linoleic and linolenic acids. Essential lipids for use in the composition can be obtained, for example, from flaxseed, soy, safflower or sesame oils.

The composition can also comprise one or more extracellular matrix components. Suitable extracellular matrix components include glucosamines, glycosaminoglycans, collagens, cartilage, chondroitin sulfates, hyaluronic acid, hyaluronan mucopolysaccharides, glycoproteins, and proteoglycans. Where shark cartilage is used, the composition can comprise about 700 mg to about 2500 mg, for example about 740 to about 1480 mg, administered 1, 2, 3, 4 or 5 times daily as part of the total composition. Shark cartilage can be obtained in powder form, and typically contains cartilage with 12% chondroitin sulfate and collagen. Suitable shark cartilage is sold under the name of Cartilade from BioTherapies, Inc., Fairfield N.J. Bovine cartilage can also be used, for example that which is available from Phoenix Biologics, Inc. (Vista, Calif.), such as administered in a dose of about 750 mg administered 1, 2, 3, 4 or 5 times daily as part of the total composition. Combinations of the shark and bovine cartilage can be used. When hyaluronic acid and hyaluronan mucopolysaccharides are used, the source may be human umbilical cord tissue.

Other suitable extracellular components include glucosamine, which is believed to be incorporated into the body's mucopolysaccharides, and hyaluronic acid, chondroitin sulfate and nutrient substrate cartilage, available from many animals including cow, pig and chicken. Suitable amounts of glucosamine for use in the composition is about 0.5 grams to about 1 gram, administered 3 times daily as part of the total composition. A suitable amount of chondroitin sulfate is about 250 mg to about 500 mg, for example 390 mg to 490 mg, administered 3 to 4 times daily as part of the total composition.

The composition can also comprise one more probiotics. Suitable probiotics include a plurality of beneficial microorganisms (such as lactobacilli, acidophilus, and other yogurt cultures), enzymes, or combinations thereof. Suitable probiotics also include any substance that promotes the growth of beneficial microorganisms in the composition or subject to which the composition is administered, either alone or in combination with other probiotics.

The composition can also comprise at lease one electrolyte, vitamin, mineral or trace element. Suitable electrolytes include sodium, potassium and calcium, and can be present in the composition in a concentration of between about 0.1% and about 50%, including any fractional percentage in intervals of about 0.01%. For example, the electrolyte (in particular, potassium) concentration can be represented as about “A.BC %,” where A is any integer selected from 0, 1, 2, 3, 4, 5, 6, 7, 8, 9, 10, 11, 12, 13, 14 ,15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50; B is any integer selected from 0, 1, 2, 3, 4, 5, 6, 7, 8, or 9; and C is any integer selected from 0, 1, 2, 3, 4, 5, 6, 7, 8 or 9. Greater or lesser amounts of electrolytes for use in the composition are contemplated. Suitable vitamins and minerals include typical adult daily dosages, for example: Vitamin A (about 1000 to about 10,000 IU; Vitamin B1 or thiamine (about 50 mg); Vitamin B2 or riboflavin (about 50 mg); Vitamin B3 as niacin or niacinamide (about 50 to about 500 mg); Vitamin B5 or pantothenic acid (about 50 to about 100 mg); Vitamin B6 or pyridoxine (about 50 m); Vitamin B12 (about 300 to about 1000 mcg); Biotin (about 300 mcg); Choline (about 100 mg); Folic acid (about 800 mcg); Inositol (about 100 mg); Para-aminobenzoic acid (about 50 mg); Vitamin C (about 50 mg to about 3000 mg or more, in multiple daily doses); Bioflavonoids (mixed—about 500 mg); Hesperidin (about 100 mg); Rutin (about 25 mg); Vitamin D (about 400 IU); Vitamin E (about 200 to about 600 IU); Vitamin K (about 100 mcg); Apatite (for example micocrystalline hydroxyapaptite—about 4762 mcg; Chromium (about 150 mcg); Copper (about 3 mg); Iodine (about 225 mcg); Iron (about 18 mg); Magnesium (about 750 to about 1,000 mg); Manganese (about 10 mg); Molybdenum (about 30 mcg); Selenium (about 200 mg); and Zinc (about 50 mg). Greater or lesser amounts of vitamins, minerals or trace elements for use in the composition are contemplated.

Thus, the anabolic composition can comprise a surfactant as described above, which can be combined with one or more of at least one amino acid, at least one extracellular matrix compound, at least one electrolyte, vitamin, mineral or trace element; and at least one probiotic.

The composition can be formulated for oral, topical or parenteral use, for example as pharmaceutical formulations. A pharmaceutical formulation comprises the composition and at least one pharmaceutically acceptable excipient, carrier or additive. Suitable topical formulations include ointments, creams or lotions, eye ointments and eye or ear drops, impregnated dressings and aerosols, and can contain conventional excipients and additives such as preservatives, solvents to assist drug penetration and emollients in ointments and creams. Topical formulations can also comprise physiologically compatible carriers, such as cream or ointment bases and ethanol or oleyl alcohol for lotions. Such carriers can be present as from about 1% up to about 98% of the formulation, for example up to about 80% of the formulation.

Oral formulations can be in the form of a compressed solid or dry powder (for example finely milled powder), which can optionally be mixed with water or other suitable liquid vehicle before use. The biologic availability of an oral formulation can be tested by placing the formulation to a vessel containing water or water and an acidic compound (such as 1-5% HCI or acetic acid) to confirm that the formulation dissolves partially or completely. Partial or complete dissolution indicates good bioavailability. For parenteral administration, fluid formulations can be prepared utilizing the therapeutic formulations of the invention mixed with a sterile, pyrogen-free physiologically acceptable carrier or excipient, such as water or physiological saline.

Colorants, flavorants, viscosity modifiers and other additives commonly used in preparing pharmaceutical or nutritional formulations can also be used, as are known to those of ordinary skill in the art. Formulations of the composition can be readily made by those of ordinary skill in the art using standard techniques, for example as described in Remington's Pharmaceutical Science, 17th ed., Mack Publishing Company, Easton, Pa. (1985), the entire disclosure of which is herein incorporated by reference.

Thus, the composition can be administered by any oral or parenteral route, for example by mouth, intrarectally, intranasally, by inhalation into the lung, intravaginally, intravascularly (by infusion or injection), intrapertioneally, intramuscularly and local administration (such as injection or deposition) in or around the tissue to be treated.

The composition can be used to treat patients suffering from (or suspected to be suffering from) a variety of chronic diseases or conditions. For example, the composition can be administered to a patient suffering from, or suspected to be suffering from cancer. The types of cancer that can be treated include cancers of at least the following histologic subtypes: sarcoma (cancers of the connective and other tissue of mesodermal origin); melanoma (cancers deriving from pigmented melanocytes); carcinoma (cancers of epithelial origin); adenocarcinoma (cancers of glandular epithelial origin); cancers of neural origin (glioma/glioblastoma and astrocytoma); and hematological neoplasias, such as leukemias and lymphomas (e.g., acute lymphoblastic leukemia and chronic myelocytic leukemia).

Types of cancers that can be treated with the composition also include cancers having their origin in any organ or tissue of the body, for example, the following organs or tissues, regardless of histologic subtype: breast; tissues of the male and female urogenital system (e.g., ureter, bladder, prostate, testis, ovary, cervix, uterus, vagina); lung; tissues of the gastrointestinal system (e.g., stomach, large and small intestine, colon, rectum); exocrine glands such as the pancreas and adrenals; tissues of the mouth and esophagus; brain and spinal cord; kidney (renal); pancreas; hepatobiliary system (e.g., liver, gall bladder); lymphatic system; smooth and striated muscle; bone and bone marrow; skin; and tissues of the eye (e.g., retinoblastomas).

Types of cancers that can be treated with the composition also include cancers or tumors in any prognostic stage of development, for example as measured by the “Overall Stage Groupings” (also called “Roman Numeral”) or the “Tumor, Nodes, and Metastases” (TNM) staging systems. Appropriate prognostic staging systems and stage descriptions for a given cancer are known in the art, for example as described in the National Cancer Institute's “CancerNet” Internet website.

An effective amount of the composition is administered to a patient suffering from (or suspected to be suffering from) cancer. An effective amount is that amount of the composition which inhibits the proliferation of a cancer cell. As used herein, to “inhibit the proliferation of a cancer cell” means to kill a cancer or tumor cell, or permanently or temporarily arrest the growth of the cell. Inhibition of tumor cell proliferation can be inferred if the number of tumor cells in the subject remains constant or decreases after administration of the composition, or cancer cell cycles and the metabolic cycles of associated organelles (such as the mitochondria) are normalized. An inhibition of cancer or tumor cell proliferation can also be inferred if the absolute number of such cells increases, but the rate of tumor growth decreases. The number of cancer cells in a subject's body can be determined by direct measurement, or by estimation from the size of primary or metastatic tumor masses. The size of a tumor mass and extent and location of metastasis can be ascertained, for example, by direct visual observation or by diagnostic imaging methods such as X-ray, magnetic resonance imaging, ultrasound, scintigraphy and PET scan. Such diagnostic imaging methods can be employed with or without contrast agents, as is known in the art. The size of a tumor mass can also be ascertained by physical means, such as palpation of the mass or measurement of the mass with a measuring instrument such as a caliper. Ascertaining the size and location of tumors or metastases can also be used to direct the focal administration of the composition, for example by direct injection to or around the tumor or metastases.

An effective amount of the composition can also comprise that amount which stops the progression of, lessens or reverses any condition or symptom associated with the cancer. For example, an effective amount can comprise an amount of the composition sufficient to stop the progression of, lessen or reverse cachexia and/or anorexia associated with a cancer (see, e.g., Examples 1 and 2 below). Indeed, an in vitro reversal of breast cancer of 76%-83% was observed after one week of intravenous administration of the composition. The prognosis of a patient with head and neck cancer also improved greatly after one week intravenous infusion of the composition. One skilled in the art can readily determine an effective amount of the composition to be administered to a patient, by taking into account factors such as the size and weight of the subject; the extent of the tumor growth or disease penetration; the age, health and sex of the subject; the route of administration; and whether the administration is regional (e.g., local) or systemic.

One skilled in the art can also readily determine an appropriate dosage regimen for administering the composition to a patient. For example, the composition can be administered to the subject once, for example as a single infusion or oral administration. Alternatively, the agent can be administered multiple times, for example once, twice, thrice, four, five or six times daily to a patient for a period of from about three to about twenty-eight days, such as from about seven to about fourteen days. In one dosage regimen, the agent is administered orally or parenterally three to four time weekly (or every other day), for three to six months or for an indefinite time period to maintain therapeutic effects.

The compositions can also be used help reduce the risks of adverse reactions associated with the use of certain allergenic plasticizers in renal dialysis, and thereby prevent recurrent anaphylaxis in dialysis and ameliorate acute flareups. Furthermore, the compositions are useful in reducing the risk of kidney or other organ or tissue transplantation rejections. Asthma may also be treated, as well as ailments of the GI tract such as regional ileitis (Crohn's Disease) and other inflammatory bowel diseases, including ulcerative colitis, mucous colitis, and liver disease such as congenital biliary atresia. The composition is particularly useful for treating inflammatory bowel diseases that are resistant to present therapies, and for treating inflammation such as that associated with atherosclerosis (e.g., peripheral vascular disease) and complications of this, which can include threatened limb loss, gangrene, coronary artery disease, myocardial infarction, stroke or cerebral vascular accident. Further diseases that can be treated with the composition include degenerative, congenital and hereditary diseases, such as congenital aneurysm (Berry aneurysm).

The composition can be used also in the treatment of trauma and deforming diseases, such as leprosy, and skin and nerve damage. Bacterial infections, such as drug resistant tuberculosis, chronic fatigue and muscle weakness can also be treated.

Diseases of the endoderm, ectoderm, mesoderm and mesenchymal surfaces can be treated with the composition. For example, diseases of the ectodermal surfaces including skin, hair, nails and teeth are amenable to treatment by the compositions. In particular, eczema, urticaria and psoriasis can be treated. The compositions can also accelerate healing and reduce the risks of corneal graft rejection.

The anabolic compositions can also reduce the effects of aging, for example when the production of digestive enzymes and growth hormone is diminished. The composition can also be used to treat immunopathies such as milk allergies, colitis, and autoimmune diseases.

Furthermore, compositions can be used to treat AIDS patients, for example those on anti-protease drugs. AIDS patients on conventional anti-protease drugs often have extreme hyperlipidemia, with serum triglyceride levels of 3,000 to 6,000 mg %. Thus, the anti-protease medication may need to be reduced or withdrawn to protect the heart and blood vessels from the medication's side-effects, such as coronary artery disease. Administration of the composition to AIDS patients on anti-protease drugs can reduce the hyperlipidemia and minimize the undesirable side effects of the drug. As a result, anti-protease dosages can be lessened while achieving the same therapeutic results.

Metabolic storage diseases, such as glycogen storage diseases lipid storage disorder, and demyelinating diseases (such as multiple sclerosis and Pelizeus-Merzbacher disease) can also be treated with the composition, as well as disorders of the blood-brain barrier and neurological diseases (e.g. rabies) and meningitis. Degenerative neurological diseases such as ALS, pernicious anemia, Alzhiemer's disease, Huntington's chorea, and prion-based diseases such as Kreutzfield-Jacob disease can also be treated with the composition. The composition can be used for preventive treatment of the diseases and conditions discussed herein.

For the diseases discussed above, the patient is administered an effective amount of the composition. An effective amount of the composition can also comprise that amount which stops the progression of, lessens or reverses any condition or symptom associated with the disease. For example, an effective amount can comprise an amount of the composition sufficient to stop the progression of, lessen or reverse inflammation associated with an inflammatory bowel disease. One skilled in the art can readily determine an effective amount of the composition to be administered to a patient, by taking into account factors such as the size and weight of the subject; the extent of disease penetration; the age, health and sex of the subject; the route of administration; and whether the administration is regional (e.g., local) or systemic. Dosage routes and dosage regimens are as described above for treatment of cancer.

Without being bound to any theory, it is thought that the essential components of the composition promote favorable substrate nutrition in vivo as well as in vitro for stem cells to thrive and participate in tissue repair, replacement and regeneration. Such effects may occur in mesodermal and mesenchymal tissue, as well as endodermal surfaces such as the gut lining and respiratory tract.

Furthermore, but without wishing to be bound by any theory, the simultaneous administration of components of the therapeutic formulations is believed to work synergistically to promote tissue healing at higher levels and at a more rapid speed than if the components were administered individually at different times.

Again without wishing to be bound by any theory, it is believed that by administering the therapeutic formulations of the present invention and avoiding or minimizing ingestion of foods containing microorganism metabolites or catabolic products (such as dairy products), the patient's recovery is enhanced because the gastrointestinal tract will only be minimally occupied in proteolysis of exogenous proteins and will still serve its immune-like functions, such as control microorganisms (an antibiotic-like function), elimination of viruses, and aiding in the repair of injured tissue to permit tissue healing through the supply of adequate nutrients.

Alternatively, and again without wishing to be bound by any theory, the composition provides nutrition to non-neoplastic cells of the patient, while depriving neoplastic cells of their primary nutrition source. Thus, the composition is advantageously used to combat the cachexia/anorexia syndrome so often seen in cancer patients.

The invention will now be illustrated with the following non-limiting examples

### EXAMPLES

### Example 1

### Treatment of Tonsillar Cancer Anal, Cancer and Small Cell Lung Cancer

At the time of treatment, Patient 1 was an approximately 35 year old male diagnosed with tonsillar cancer. His prognosis was determined to be poor. At the time of treatment, Patient 2 was an 81 year old male diagnosed with anal cancer having local dissemination. At the time of treatment, Patient 3 was an approximately 70 year old female diagnosed with intractable small cell lung cancer, with metastasis and cachexia. Patient 3 weighed approximately 90 lbs and complained of fatigue.

Patient 2 received a sterile non-pyrogenic composition of about 20% essential fatty acids (a mixture of linoleic and linolenic acid), 1.2% egg yolk phospholipid, and about 2% glycerin in water for injection. This composition is sold commercially as “Intralipid,” and was obtained from Baxter Healthcare Corp. (Deerfield, Ill.); see attached photocopy of Intralipid IV package. Intralipid is manufactured for Baxter Healthcare by Fresenius Kabi AB (Uppsala, Sweden). Five hundred milliliters of Intralipid was given intravenously three times a week for one week. Patients 1 and 3 received a similar composition called Liposyn (Abbot Laboratories) intravenously three times a week for one week. Patient 2 received 500 ml per dose and Patient 3 was administered only 250 ml per dose. Liposyn is a sterile, nonpyrogenic fat emulsion for intravenous administration, which contains about 10% safflower oil, 1.2% egg phosphatides and about 2.5% glycerin in water for injection.

Patients 1-3 showed an improvement in overall heath, including an increase in energy and relief of the fatigue and listlessness characteristic of cancer and disseminated cancer. Clinical signs of disease were visibly lessened. Regarding Patient 1, the prognosis was so grave that any signs of recovery were unexpected. However, after one week of treatment, it was reported that the improvement in Patient 1 was so dramatic that heath-care personnel had to verify the patient's name to be sure he was the same patient that had been admitted. Patient's 1 improvement continued throughout the entire course of treatment. Patient 2 was observed walking down the hall one month after treatment, and his gait was remarkably brisk in view of his age, and the extent and location of his anal cancer. Patient 3 suffered from the fatigue characteristic of cancer, particularly when complicated by metastasis and cachexia. However, 24 hours after administering the composition to Patient 3, her fatigue completely resolved, which was a completely unexpected result in a patient with such a poor prognosis. This complete clearance of fatigue was noted again, lasting for 24 hours, after administration of the next two doses.

### Example 2

### Treatment of Squamous Cell Cancer, Lung Adenocarcinoma, Metastatic Lung Cancer and Anal Cancer

Patient 4 is a 57-year old male with squamous cell cancer of the hypopharynx. Within one week, this patient was given a single dose of 500 ml Intralipid (20%) and a single dose of 500 ml Intralipid (10%). After this treatment, the patient showed some clinical improvement in energy levels and a decrease in lassitude.

Patient 5 is a 74-year old female with adenocarcinoma of the lung. This patient received three 500 ml doses of Intralipid (20%) in one week, and showed moderately increased energy levels and a reduction in lassitude.

Patient 6 is an 81-year old male suffering from right upper lobe lung cancer with metastatic spread in cervical lymph nodes right to left. This patient received one-daily doses of 250 ml Intralipid (10%) for 3 consecutive days. Patient 6 showed marked improvement in energy levels and a more than 60% lessening of radiation treatment side effects.

Patient 7 is an adult male, 82-years old, suffering from anal cancer complicated by severe cachexia. At the time of treatment, this patient weighed approximately 144 lbs. Upon 3 times weekly dosing with 500 ml Intralipid (20%), the size of a superficial tumor in the anaUperianal area (initially 5 cm) was reduced 40% in size after three days of treatment. After five treatments, the enlarged inguinal lymph nodes returned to normal. One indication that local dissemination of the cancer had improved.

From clinical observations of these patients before, during and after treatment with the Intralipid and Liposyn compositions, it is believed that the improvement in their clinical condition was the direct result of administering the composition.

### Example 3

### Treatment of Crohn's Disease

A 73 year old female patient suffering from Crohn's disease (symptoms included diarrhea, constipation, severe bouts of abdominal pain and fever, G.I. bleeding, generalized aching, extreme fatigue, nausea, and food and dairy intolerance) was being treated with corticosteroids administered three times weekly. This patient was administered a composition comprising about 10.6 g Neocate infant formula containing L-amino acids and glycine; about 50-100 mg lecithin; about 12.5-40 mg phosphatidyl choline; about 1000 mg fish oil concentrate (Entero-coated Fish Oil, 180 mg EPA and 120 mg DHA, Leiner Health Products, LLC, Carson Calif.); VSL (Biffidum bacterium breve, Lactobacillus acidophilus, B. bacterium longum, L. plantarum, B. bacterium infantis, L. baracaciae, Streptococcus thermophilus, L. bulgaricus) and/or Digestive Formula two-phase digestive aid available from Life Plus International, Batesville, Ak. (1 tablet daily, contains pancreatin, pancreolipase, pepsin, amylase, papain, bromelain and lipase, betaine, lactobacillus microflora such as L. salivarius, L. acidophilus, L. dds-1, L. bulgaricus and Biffidum bacteria, bile, lecithin, peppermint leaf, aloe vera and beetroot), extracellular matrix components comprising collagen, proteoglycan aggregate complex of cartilage and chondroitin sulfate (bovine and/or shark cartilage, four 740 mg capsules, twice or more daily), 3 mg boron, micocrystalline hydroxyapaptite (4762 mcg supplied as “Boneup” from Jarrow Formulas, Los Angeles, Calif., of which 1000 mcg is Ca, 510 mcg is P, and 1514 mcg is protein, 500 mg magnesium oxide, 10 mg zinc monomethionate, 1 mg copper gluconate, 1 mg manganese citrate, 300 mg glucosamine, 200 mg Vitamin C, 500 IU of Vitamin D3, 100 mcg Vitamin K as menaquinone-7, 400 mcg folic acid, and 100 mcg Vitamin B12), 200 mcg selenium, an additional 500-1000 mcg Vitamin B12, and 5 ml (preferably at bedtime) daily to three times a week of cherry flavored potassium chloride oral solution U.S.P. 10% (HUMCO, Texarkana, Tx. 75501), which contains 20 mEq (1.5 g) of potassium chloride. At times, the patient was given the potassium chloride solution as much as 5 ml three times a day as much 15 ml two to three times a day. Clinical observation showed a amelioration of severe abdominal pain and diarrhea, a decrease in fatigue, and control of osteoporosis caused by steroid use, age and previous ovarectomy. Complete normality of sedimentation rate and C-reactive proteins was also observed. A recent mild myocardial infarction has prompted the addition of three 900 mg arginine capsules in addition to the above-listed components which, along with nitroglycerin patches, has served to reduce angina. A goiter present in the patient's neck has also been maintained at a manageable size, which has precluded the need for surgical removal of the goiter. It should be noted that, at time, the VSL is not swallowed, but only used as a mouth rinse because it can aggravate the patient's diarrhea. While the present invention has been described in connection with the described ents, it is understood that other similar embodiments may be used or modifications and s made to the described embodiments for performing the same function without g therefrom. Therefore, the invention should not be limited to any single embodiment, er should be construed in breadth and scope in accordance with the recitation of the appended claims.

I claim:

1. An anabolic composition, comprising at least one amino acid, at least one extracellular matrix compound, and at least one surfactant, wherein the concentration of surfactant in the composition is about 1% or greater (w/w or w/v) with respect to the total composition.

2. The anabolic composition of claim 1, wherein the at least one surfactant is a lipid.

3. The anabolic composition of claim 2, wherein the lipid is a phospholipid or essential lipid.

4. The anabolic composition of claim 3, wherein the lipid is selected from the group consisting of phosphatidylcholine; phosphatidylserine; phosphatidylinositol; phosphatidylethanolamine; phosphatidic acid; phosphotidyl glycerol sphingolipids; sphingomyelin; glycolipids; cerbrosides; gangliosides; cephalin; lipovitellin; glycosphingolipids; lipids containing linoleic or linolenic acids; EPA; DHA and combinations thereof.

5. The anabolic composition of claim 1, wherein the at least one surfactant is selected from the group consisting of glycerol monostearate; diethylene glycol fatty acid ester; polyoxyethylene sorbitol beeswax derivative; diethylene glycol monolaurate; diethylene glycol fatty acid ester; polyoxyethylene dioleate; sorbitan monopalmitate; sorbitan monolaurate; tetraethylene glycol monostearate; tetraethylene glycol monooleate; polyoxypropylene mannitol dioleate; polyoxyethylene sorbitol lanolin oleate derivative; polyoxypropylene stearate; polyoxyethylene fatty acid; polyoxyethylene sorbitol beeswax derivative; polyoxyethylene sorbitan monostearate; polyoxyethylene sorbitan monooleate; polyoxyethylene oxypropylene oleate; polyoxyethylene cetyl ether; polyoxyethylene sorbitan tristearate; polyoxyethylene lauryl ether; tetraethylene glycol monolaurate; polyoxyethylene lauryl ether; polyoxyethylene sorbitan trioleate; hexaethylene glycol monostearate; polyoxyethylene esters of mixed fatty and resin acids; polyoxyethylene oxypropylene oleate; polyoxyethylene lanolin derivative; polyoxyethylene monostearate; polyoxyethylene monopalmitate; alkyl aryl sultanate; triethanolamine oleate; polyoxyethylene alkyl phenol; polyoxyethylene sorbitol lanolin derivative; polyoxyethylene alkyl aryl ether; polyoxyethylene castor oil; polyoxyethylene vegetable oil; polyoxyethylene oleyl ether; polyoxyethylene stearyl alcohol; polyoxyethylene oleyl alcohol; polyoxyethylene fatty alcohol; polyoxyethylene cetyl alcohol; polyoxyethylene glycol monopalmitate; polyoxyethylene sorbitan monopalmitate; polyoxyethylene oxypropylene stearate; sodium oleate; potassium oleate; N-cetyl-N-ethyl morpholinium; polyglycerol polyricinolate; polysorbate 80; polysorbate 65 and sodium lauryl sulfate.

6. The anabolic composition of claim 1, wherein the at least one surfactant is selected from the group consisting of lipid, a phospholipid, a glycolipid, a monoglyceride, a diglyceride and a lipoprotein.

7. The anabolic composition of claim 1, wherein the at least one surfactant is present in a concentration of between about 1.2% and about 20%.

8. The anabolic composition of claim 1, wherein the at least one surfactant is present in a concentration selected from the group consisting of about 1.2%, about 1.3%, about 1.4%, about 1.5%, about 1.6%, about 1.7%, about 1.8%, about 1.9%, about 2%, about 3%, about 4%, about 5%, about 10% and about 15%.

9. The anabolic composition of claim 1, wherein the extracellular matrix compound is selected from the group consisting of glucosamines, glycosaminoglycans, collagens, cartilage, chondroitin sulfates, hyaluronic acid, hyaluronan mucopolysccharides, glycoproteins, and proteoglycans.

10. The anabolic composition of claim 1, further comprising at least one electrolyte, mineral, vitamin, trace element, or combinations thereof.

11. The anabolic composition of claim 10, wherein the electrolyte comprises potassium ions.

12. The anabolic composition of claim 1, further comprising a probiotic.

13. A method of treating a disease or condition in a subject, comprising:
(1) providing a subject who has the disease or condition;
(2) administering to the subject an effective amount of a composition comprising at least one surfactant, wherein the total concentration of surfactant in the composition is greater than 1% (w/w or w/v).

14. The method of claim 13, wherein the at least one surfactant is a lipid.

15. The method of claim 14, wherein the lipid is a phospholipid or essential lipid.

16. The method of claim 15, wherein the lipid is selected from the group consisting of phosphatidylcholine; phosphatidylserine; phosphatidylinositol; phosphatidylethanolamine; phosphatidic acid; phosphotidyl glycerol sphingolipids; sphingomyelin; glycolipids; cerbrosides; gangliosides; cephalin; lipovitellin; glycosphingolipids; lipids containing linoleic or linolenic acids; EPA; DHA and combinations thereof.

17. The method of claim 13, wherein the at least one surfactant is selected from the group consisting of glycerol monostearate; diethylene glycol fatty acid ester; polyoxyethylene sorbitol beeswax derivative; diethylene glycol monolaurate; diethylene glycol fatty acid ester; polyoxyethylene dioleate; sorbitan monopalmitate; sorbitan monolaurate; tetraethylene glycol monostearate; tetraethylene glycol monooleate; polyoxypropylene mannitol dioleate; polyoxyethylene sorbitol lanolin oleate derivative; polyoxypropylene stearate; polyoxyethylene fatty acid; polyoxyethylene sorbitol beeswax derivative; polyoxyethylene sorbitan monostearate; polyoxyethylene sorbitan monooleate; polyoxyethylene oxypropylene oleate; polyoxyethylene cetyl ether; polyoxyethylene sorbitan tristearate; polyoxyethylene lauryl ether; tetraethylene glycol monolaurate; polyoxyethylene lauryl ether; polyoxyethylene sorbitan trioleate; hexaethylene glycol monostearate; polyoxyethylene esters of mixed fatty and resin acids; polyoxyethylene oxypropylene oleate; polyoxyethylene lanolin derivative; polyoxyethylene monostearate; polyoxyethylene monopalmitate; alkyl aryl sultanate; triethanolamine oleate; polyoxyethylene alkyl phenol; polyoxyethylene sorbitol lanolin derivative; polyoxyethylene alkyl aryl ether; polyoxyethylene castor oil; polyoxyethylene vegetable oil; polyoxyethylene oleyl ether; polyoxyethylene stearyl alcohol; polyoxyethylene oleyl alcohol; polyoxyethylene fatty alcohol; polyoxyethylene cetyl alcohol; polyoxyethylene glycol monopalmitate; polyoxyethylene sorbitan monopalmitate; polyoxyethylene oxypropylene stearate; sodium oleate; potassium oleate; N-cetyl-N-ethyl morpholinium; polyglycerol polyricinolate; polysorbate 80; polysorbate 65 and sodium lauryl sulfate.

18. The method of claim 13, wherein the lipid is a polar surface active lipid.

19. The method of claim 13, wherein the at least one surfactant is selected from the group consisting of lipid, a phospholipid, a glycolipid, a monoglyceride, a diglyceride and a lipoprotein.

20. The method of claim 13, wherein the at least one surfactant is present in a concentration of between about 1.2% and about 20%.

21. The method of claim 13, wherein the at least one surfactant is present in a concentration selected from the group consisting of about 1.2%, about 1.3%, about 1.4%, about 1.5%, about 1.6%, about 1.7%, about 1.8%, about 1.9%, about 2%, about 3%, about 4%, about 5%, about 10% and about 15%.

22. The method of claim 13, wherein the extracellular matrix compound is selected from the group consisting of glucosamines, glycosaminoglycans, collagens, cartilage, chondroitin sulfates, hyaluronic acid, hyaluronan mucopolysccharides, glycoproteins, and proteoglycans.

23. The method of claim 13, wherein the composition further comprises at least one electrolyte, mineral, vitamin, trace element, or combinations thereof.

24. The method of claim 23, wherein the electrolyte comprises potassium ions.

25. The method of claim 13, wherein the composition further comprises a probiotic.

26. The method of claim 13, wherein the disease is cancer.

27. The method of claim 26, wherein the cancer is selected from the group consisting of breast cancer; cervical cancer; tonsillar cancer, anal cancer; small cell lung cancer; squamous cell cancer; lung adenocarcinoma, and metastatic lung cancer.

28. The method of claim 13, wherein the disease or condition is selected from the group consisting of AIDS; asthma; cachexia; anorexia; eczema; urticaria; psoriasis; leprosy; skin and nerve damage; bacterial infections; inflammatory bowel disease; liver disease; coronary artery disease; peripheral artery disease; cerebral vascular disease; atherosclerosis; chronic fatigue; muscle weakness; and metabolic storage diseases.

29. The method of claim 28, wherein the inflammatory bowel disease is regional ileitis (Crohn's Disease); ulcerative colitis; or mucous colitis.
